(19) United States
(12) Patent Application Publication
(10) Pub. No.: US 2024/0343696 A1
(43) Pub. Date: Oct. 17, 2024

(54) COMPOUNDS AND USES THEREOF IN THE TREATMENT AND PREVENTION OF DISEASES AND CONDITIONS ASSOCIATE WITH OR AGGRAVATED BY IMPAIRED MITOPHAGY OR OXIDATIVE STRESS

(71) Applicant: Yissum Research Development Company of The Hebrew University of Jerusalem Ltd., Jerusalem (IL)

(72) Inventors: Shmuel BEN-SASSON Z”L, Jerusalem (IL); Einav GROSS, Jerusalem (IL)

(73) Assignee: Yissum Research Development Company of The Hebrew University of Jerusalem Ltd., Jerusalem (IL)

(21) Appl. No.: 18/576,379

(22) PCT Filed: Jul. 5, 2022
(86) PCT No.: PCT/IL2022/050722

§ 371 Date: Jan. 4, 2024

Related U.S. Application Data
(60) Provisional application No. 63/218,420, filed on Jul. 5, 2021.

(51) Int. Cl. C07D 249/12; A61K 31/4155; A61K 31/4178; A61K 31/4192; A61K 31/4196; A61P 35/00; A61P 39/00; C07D 231/12; C07D 231/18; C07D 233/64; C07D 233/84; C07D 249/04

(52) U.S. Cl. CPC C07D 249/12; A61K 31/4155; A61K 31/4178; A61K 31/4192; A61K 31/4196; A61P 35/00; A61P 39/00; C07D 231/12; C07D 231/18; C07D 233/64; C07D 233/84; C07D 249/04

(57) ABSTRACT

The invention provides compounds and uses thereof in the treatment and prevention of diseases and conditions associate with or aggravated by impaired mitophagy or oxidative stress, and methods of their preparation.

### BACKGROUND OF THE INVENTION

Mitochondria (MT) are double-membrane-bound organelles found in most eukaryotic organisms. They are essential for chemical energy production, in the form of ATP, in all aerobic organisms, including humans. Moreover, mitochondria are essential for many other metabolic processes including the synthesis of amino acids, lipids, heme, steroid hormones, and are the source for reactive oxygen species (ROS).

ROS present cells with a double-edged sword. On the one hand, they play a crucial role in many cellular and physiological processes including the innate immune response and the degradation and recycling of the cellular milieu in a process called autophagy. On the other hand, ROS interact with metals to produce toxic oxygen (O₂) radicals that can damage DNA and biological membranes and thus interfere with mitochondria function and cause cell injury and death. Mitochondria themselves are generating ROS, as part of their physiological activity, and are especially vulnerable to ROS-induced damage. Therefore, to maintain healthy mitochondria there is a constant need to generate new mitochondrial components (mitochondrial biogenesis) while removing the damaged one by a process known as mitophagy (=mitochondrial-autophagy).

The proper functioning of this intracellular “quality-control” mechanism of mitophagy is especially important in tissues where no renewal by cell-division is taking place. Cells of nonrenewable tissues include neurons, skeletal muscle and heart muscle cells, insulin-producing beta-cells of the endocrine pancreas, cells of the retinal pigment epithelium and more. Indeed, degenerative diseases associated with aging belong mainly to such nonrenewable tissue including dementia, Alzheimer's and Parkinson's diseases, sarcopenia (=skeletal muscle atrophy), congestive heart failure, type 2 diabetes, age-related macular degeneration, fibrosis, including lung-fibrosis, and more.

While the biogenesis of mitochondria does not generally decline with age (and may even increase), mitophagy is profoundly decreased. Therefore, the accumulation of damaged mitochondria is thought to underlie the decline in organ function and health span. The current consensus is that impaired mitophagy plays a pivotal role in the development of these degenerative diseases associated with aging (Markaki M. et al. Int Rev Cell Mol Biol (2018) 340: 169-208).

It was shown that subjects with Parkinson's disease (PD) have compromised mitophagy processes (Lee S H et al. (2016) EMBO Mol Med 8:779-85; Gao F. et al. Frot Neurol (2017) 8:527). Indeed, mitochondrial dysfunction appears to be a key factor in the pathophysiology of both familial and sporadic PD as well as in cases of toxin-induced Parkinsonism (Rayn B J. et al. Trends Biochem Sci (2015) 40:200-10).

Inadequate mitophagy leads to excessive ROS formation. Therefore, mitophagy links oxidative stress conditions and neurodegenerative diseases (Shefa U. et al. Neural Regen Res (2019) 14:749-756). Thus, the terms “impaired mitophagy” and “oxidative stress” or “oxidative injury” are hereby used interchangeably to describe unfavorable conditions that lead to the evolvement of aging-associated diseases.

The toxin 1-methyl 4-phenyl 1,2,3,6-tetrahydropyridine (MPTP) induces Parkinsonian syndrome in people (Langston W J. et al. Science (1983) 219: 979-980). Since the chemical structures MPTP and the pesticide N,N′-dimethyl-4,4′-bipyridinium dichloride (paraquat) are similar, paraquat is widely used in animal model of PD (Miller G W. Toxicol Sci (2007) 100: 1-2). Moreover, paraquat (PQ) is a robust inducer of oxidative stress in cells (Halliwell B., Gutteridge J. in Free Radicals in Biology and Medicine, Clarendon Press, Oxford, 2006). Therefore, induction of mitophagy should increase the resistance to PQ-induced oxidative injury. In agreement with the above, it was shown that compromising C. elegans mitophagy make this organism more vulnerable to PQ toxicity (Luz A L et al. Toxicology (2017) 387:81-94).

Thus, it is well established that the protection of cells and organisms against paraquat-induced damage is a hallmark of mitophagy augmentation (Dagda R A et al. Int. J. Mol. Sci. 14: 22163-89 (2013)).

There is a need for a medicaments and methods capable of treating and/or protecting the human body from the damages of impaired mitophagy/mitochondrial-autophagy and oxidative injury, including any conditions, diseases, disorders and symptoms associated therewith and also including conditions and diseases associated with cell degeneration, in particular in cells of non-regenerative tissues.

### SUMMARY OF THE INVENTION

The present invention provides a compound having a general formula (I);

R₁—X₁-L-X₂—R₂ (I)
wherein R₁ and R₂ are each independently selected from —C(═NR₃)NR₄R₅, —NR₆R₇, —N⁺R₈R₉R₁₀, —NR₁₁C(═N)NR₁₂R₁₃, —NR₁₈NR₁₉R₂₀, —ONR₂₂R₂₃, —NR₁₄C(═N)—NR₁₅—C(═N)—NR₁₆R₁₇═N—R₂₁

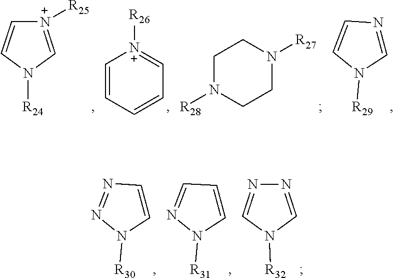

wherein each of R₃-R₃₂ is independently selected from H, straight or branched C₁-C₁₂ alkyl, straight or branched C₂-C₁₂ alkenyl, straight or branched C₂-C₁₂ alkynyl, phenyl, —OH, halogen and any combinations thereof;
L is selected from straight or branched C₄-C₁₂ alkylene, straight or branched C₄-C₁₂ alkenylene, straight or branched C₄-C₁₂ alkynylene;
each L is optionally interrupted by at least one of C₄-C₈ cycloalkylene, C₄-C₈ cycloalkenylene, C₄-C₈ cycloalkynylene, arylene, heteroarylene, heteroatom and any combinations thereof;
each L is optionally substituted with at least one of halogen and any combinations thereof;
X₁ and X₂ are each independently selected from null, —O—, —S—, —S(═O)—, —S(═O)₂—
provided that when X₁ and X₂ are each selected from null, —O—, —S— than R₁ and R₂ are independently selected from

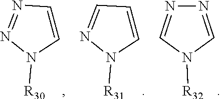

In some embodiments, L is straight or branched C₄-C₁₂ alkylene. In some embodiments, L is straight or branched C₄-C₈ alkylene. In some embodiments, L is straight or branched C₄-C₁₂ alkylene. In some embodiments, L is straight or branched C₁₀-C₁₂ alkylene. In some embodiments, L is straight or branched C₄ alkylene. In some embodiments, L is straight or branched C₅ alkylene. In some embodiments, L is straight or branched C₆ alkylene. In some embodiments, L is straight or branched C₇ alkylene. In some embodiments, L is straight or branched C₈ alkylene. In some embodiments, L is straight or branched C₉ alkylene. In some embodiments, L is straight or branched C₁₀ alkylene. In some embodiments, L is straight or branched C₁₁ alkylene. In some embodiments, L is straight or branched C₁₂ alkylene.

It should be understood that the term “interrupted by” as used herein refers to the option wherein at least one moiety as listed herein above is connected between any two carbon atoms of L, thus said at least one moiety has two open valencies. Furthermore, the term “substituted with” should be understood to relate to the option of substituting at least one hydrogen atom of L with at least one moiety as listed herein above, thus said at least one moiety has one open valency.

In some embodiments, L is interrupted by at least one of C₄-C₈ cycloalkylene, C₄-C₈ cycloalkenylene, C₄-C₈ cycloalkynylene, aryl, heteroaryl, heteroatom and any combinations thereof. In other embodiments, L is interrupted by at least one C₄-C₈ cycloalkylene. In further embodiments, L is interrupted by at least one C₄-C₈ cycloalkenylene. In some embodiments, L is interrupted by at least one C₄-C₈ cycloalkynylene. In some embodiments, L is interrupted by at least one aryl selected from phenyl or biphenyl. In some embodiments, L is interrupted by at least one heteroaryl. In some embodiments, L is interrupted by at least one heteroatom selected from N, O, S. In some embodiments, L is substituted with at least one of halogen selected from F, Br, Cl, I and any combinations thereof.

In some embodiments, R₁ and R₂ are identical. In other embodiments, R₁ and R₂ are different. In some embodiments, X₁ and X₂ are identical. In other embodiments, X₁ and X₂ are different. In some embodiments, at least one of X₁ and X₂ is null.

In further embodiments each of X₁ and X₂ is selected from —O— and —S—; wherein R₁ and R₂ are each selected from

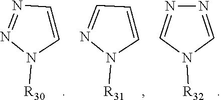

In some embodiments, R₁ and R₂ are each —C(═NR₃)NR₄R₅. In some embodiments, R₁ and R₂ are each selected from —NR₆R₇ and —N⁺R₈R₉R₁₀. In some embodiments, R₁ and R₂ are each selected from —NR₁₁C(═N)NR₁₂R₁₃ and —NR₁₄C(═N)—NR₁₅—C(═N)—NR₁₆R₁₇. In some embodiments, R₁ and R₂ are each —NR₁₈NR₁₉R₂₀. In some embodiments, R₁ and R₂ are each ═N—R₂₁. In some embodiments, R₁ and R₂ are each —ONR₂₂R₂₃. In some embodiments, R₁ and R₂ are each

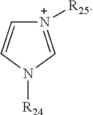

In some embodiments, R₁ and R₂ are each

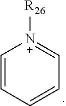

In some embodiments, R₁ and R₂ are each

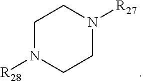

In some embodiments, R₁ and R₂ are each

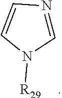

In some embodiments, R₁ and R₂ are each

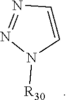

In some embodiments, R₁ and R₂ are each

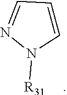

In some embodiments, R₁ and R₂ are each

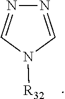

In some embodiments, X₁ and X₂ are each independently selected from null, —O—, —S— and any combinations thereof. In some embodiments, X₁ and X₂ are each independently selected —S(═O)—, —S(═O)₂— and any combinations thereof.

In further embodiments each of X₁ and X₂ is selected from —S(═O)— and —S(═O)₂—.

In some other embodiments, R₁ and R₂ are each selected from

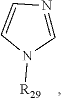

In some embodiments, a compound of the invention is selected from:

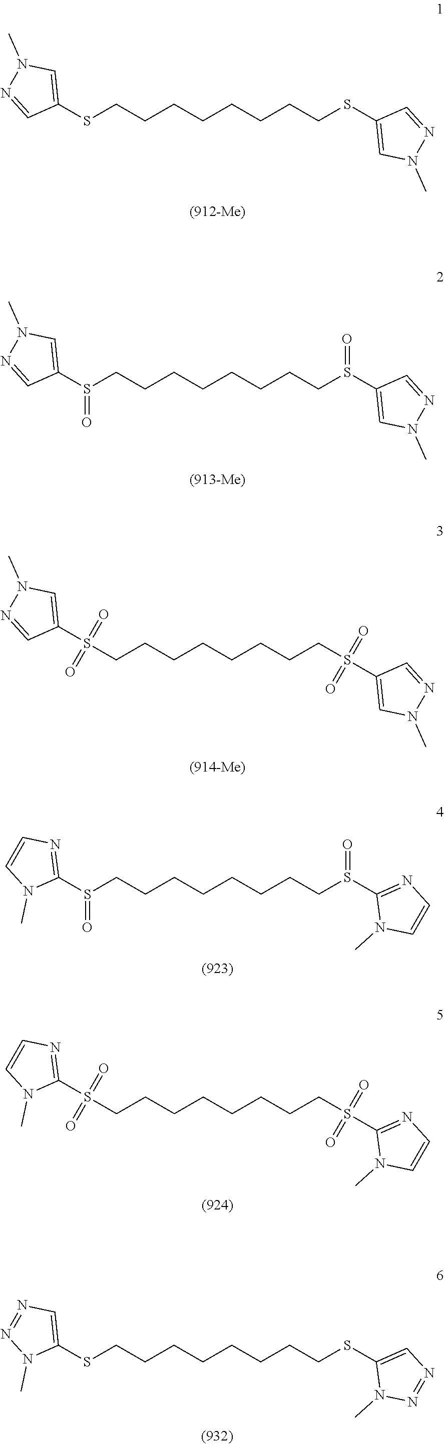 [SMILES: Cn1cc(SCCCCCCCCSc2cnn(C)c2)cn1 ; Cn1cc(S(=O)CCCCCCCCS(=O)c2cnn(C)c2)cn1 ; Cn1cc(S(=O)(=O)CCCCCCCCS(=O)(=O)c2cnn(C)c2)cn1 ; Cn1ccnc1S(=O)CCCCCCCCS(=O)c1nccn1C ; Cn1ccnc1S(=O)(=O)CCCCCCCCS(=O)(=O)c1nccn1C ; Cn1nncc1SCCCCCCCCSc1cnnn1C]

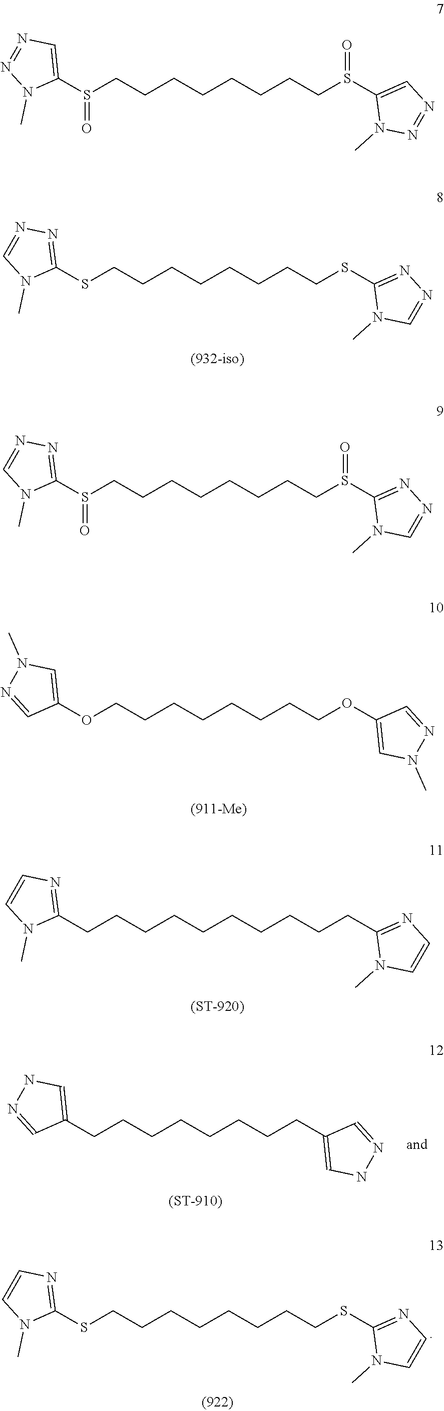

In another aspect the invention provides a composition comprising at least one compound as defined herein above.

In further aspect, the invention provides a compound as defined herein above, for use it the treatment of a condition or a disease associated with cell degeneration.

When referring to “treatment of a disease, disorder, symptom, which is caused by, associated with, or aggravated by impaired mitophagy” it should be understood to encompass the management and care of a patient for the purpose of combating a disease, disorder, condition or symptom and includes the slowing the progression or delaying of the progression of the disease, disorder, condition or symptom, the alleviation or relief of symptoms and complications, and/or the cure or elimination of the disease, disorder or condition. Said condition, disease, disorder or symptom are defined to be associated with directly or indirectly, caused by directly or indirectly or directly or indirectly aggravated by impaired mitophagy process, i.e. the cellular process of removing damaged mitochondria is biologically inefficient, reduced and insufficient for the purposes of maintaining a healthy viable cell. In some embodiments, said mitophagy process is a process in cells of non-regenerative tissues.

When referring to “prevention of a disease, disorder, symptom, which is caused by, associated with, or aggravated by impaired mitophagy” it should be understood to encompass to substantially stopping the occurrence or progression of a disease, disorder, condition or symptom. Said condition, disease, disorder or symptom are defined to be associated with directly or indirectly, caused by directly or indirectly or directly or indirectly aggravated by impaired mitophagy process, i.e. the cellular process of removing damaged mitochondria is biologically inefficient, reduced and insufficient for the purposes of maintaining a healthy viable cell. In some embodiments, said mitophagy process is a process in cells of non-regenerative tissues.

When relating to the use of compounds of the invention in the “treatment of a condition, disease, disorder or symptom associated with cell degeneration”, it should be understood to relate to the management and care of a patient for the purpose of combating a disease, disorder, condition or symptom and includes the prevention or delaying of the progression of the disease, disorder, condition or symptom, the alleviation or relief of symptoms and complications, and/or the cure or elimination of the disease, disorder or condition. Said condition, disease, disorder or symptom are defined to be associated with, caused by, or aggravated by the process of inexorable slide into no functionality of cells caused by stochastic degradation of its parts, in some embodiments the mitochondria. In further embodiments, the invention is directed to the treatment of conditions, disorders, diseases or symptoms associated with cell degeneration of non-regenerative tissues. Such “non-regenerative tissue” include tissues that do not spontaneously regenerate such as neurons (central and peripheral nervous system), cardiomyocytes (heart muscle cells), skeletal-muscle cells, insulin-producing cells (beta-cells of the endocrine pancreas), and retinal pigment epithelium.

In further aspect, the invention provides a compound as defined herein above, for use it the slowing the progression of or preventing a condition or a disease associated with cell degeneration.

When referring to “slowing the progression” it should be understood to relate to delaying of the progression of the disease, disorder, condition or symptom, associated with, caused by, or aggravated by cell degeneration. In some embodiments, the invention is directed to the treatment of conditions, disorders, diseases or symptoms associated with cell degeneration of non-regenerative tissue.

When referring to “preventing” it should be understood to relate to substantially stopping the occurrence or progression of the disease, disorder, condition or symptom, associated with, caused by, or aggravated by cell degeneration. In some embodiments, the invention is directed to the treatment of conditions, disorders, diseases or symptoms associated with cell degeneration of non-regenerative tissue.

In some embodiments, said condition or a disease associated with cell degeneration is a neurodegenerative disease, disorder and condition associated therewith.

In other embodiments, said condition or a disease associated with cell degeneration is an age-related disease, disorder and condition associated therewith.

In further embodiments, said condition or a disease associated with cell degeneration is selected from Parkinson's disease, Alzheimer's disease, dementia, congestive heart failure, sarcopenia, type 2 diabetes, age-related macular degeneration (AMD), atherosclerosis, cardiovascular diseases, cancer, liver diseases, pancreatic diseases, ocular diseases, arthritis, cataracts, osteoporosis, hypertension, fibrosis, including lung-fibrosis, and any combinations thereof.

The invention further provides a compound as defined herein above and below for use in a method of maintaining the vitality of non-regenerating tissue in a subject, said method comprising administering to said subject an effective dose of a compound as defined herein above and below.

When referring to “maintaining the vitality of non-regenerating tissue” it should be understood to relate to keeping the vital state of a non-regenerating tissue by slowing down the progression or preventing said tissue cell degeneration. Upon maintaining the vitality of non-regenerative tissue, the lifespan of a subject treated with a compound of the invention can be prolonged.

The invention further provides a method of maintaining the vitality of non-regenerating tissue in a subject, said method comprising administering to said subject an effective dose of a compound as defined herein above and below.

The invention further provides a method for the treatment of a condition or a disease associated with cell degeneration in a subject, said method comprising administering to said subject an effective dose of a compound as defined herein above and below.

The invention further provides a method for slowing the progression of or preventing a condition or a disease associated with cell degeneration in a subject, said method comprising administering to said subject an effective dose of a compound as defined herein above and below.

In further aspect, the invention provides a compound as defined herein above, for use in facilitating mitophagy and preventing oxidative injury. When referring to the facilitation of mitophagy or preventing oxidative injury it should be understood to encompass the promotion of, enhancement of, enablement of the process of mitophagy in cells or preventing oxidative injury, thereby prolonging the viability of said cells. In some embodiments, said cells are of non-regenerative tissue.

In further aspect, the invention provides a compound as defined herein above, for use in facilitating mitophagy in cancer cells, in order to promote their death. For example, it was shown that in hepatocellular carcinoma (HCC, known also as liver cancer), triggering mitophagy, will result in enhanced HCC apoptosis (for a brief review see: Aman Y. et al. Iron out, mitophagy in! A way to slow down hepatocellular carcinoma. EMBO Reports (2020) 21: e51652).

The term “straight or branched C₁-C₁₂ alkyl” should be understood to encompass any straight or branched saturated hydrocarbon chain having 1, 2, 3, 4, 5, 6, 7, 8, 9, 10, 11 or 12 carbon atoms, wherein only sigma bonds connect between the atoms of the chain, and wherein one hydrogen atom is removed from any carbon atom of the chain.

The term “straight or branched C₂-C₁₂ alkenyl” should be understood to encompass any straight or branched unsaturated hydrocarbon chain having 2, 3, 4, 5, 6, 7, 8, 9, 10, 11 or 12 carbon atoms, wherein at least one double bond connects two carbon atoms at any point of the hydrocarbon chain, and wherein one hydrogen atom is removed from any carbon atom of the chain.

The term “straight or branched C₂-C₁₂ alkynyl” should be understood to encompass any straight or branched unsaturated hydrocarbon chain having 2, 3, 4, 5, 6, 7, 8, 9, 10, 11 or 12 carbon atoms, wherein at least one triple bond connects two carbon atoms at any point of the hydrocarbon chain, and wherein one hydrogen atom is removed from any carbon atom of the chain.

The term “straight or branched C₄-C₁₂ alkylene” should be understood to encompass any straight or branched saturated hydrocarbon chain having 4, 5, 6, 7, 8, 9, 10, 11 or 12 carbon atoms, wherein only sigma bonds connect between the atoms of the chain, and wherein two hydrogen atoms are removed from any two carbon atoms of the chain.

The term “straight or branched C₄-C₁₂ alkenylene” should be understood to encompass any straight or branched unsaturated hydrocarbon chain having 4, 5, 6, 7, 8, 9, 10, 11 or 12 carbon atoms, wherein at least one double bond connects two carbon atoms at any point of the hydrocarbon chain, and wherein two hydrogen atoms are removed from any two carbon atoms of the chain.

The term “straight or branched C₄-C₁₂ alkynylene” should be understood to encompass any straight or branched unsaturated hydrocarbon chain having 4, 5, 6, 7, 8, 9, 10, 11 or 12 carbon atoms, wherein at least one triple bond connects two carbon atoms at any point of the hydrocarbon chain, and wherein two hydrogen atoms are removed from any two carbon atoms of the chain.

The term “C₄-C₈ cycloalkylene” should be understood to encompass any saturated cyclic hydrocarbon ring having 4, 5, 6, 7, 8, carbon atoms, wherein only sigma bonds connect between the atoms of the ring, and wherein two hydrogen atoms are removed from any carbon atoms of the ring.

The term “C₄-C₈ cycloalkenylene” should be understood to encompass any cyclic unsaturated hydrocarbon ring having 4, 5, 6, 7, 8 carbon atoms, wherein at least one double bond connects two carbon atoms at any point of the hydrocarbon ring, and wherein two hydrogen atoms are removed from any two carbon atoms of the ring.

The term “C₄-C₈ cycloalkynylene” should be understood to encompass any cyclic unsaturated hydrocarbon ring having 4, 5, 6, 7, 8 carbon atoms, wherein at least one triple bond connects two carbon atoms at any point of the hydrocarbon ring, and wherein two hydrogen atoms are removed from any two carbon atoms of the ring.

As used herein, the term “arylene” refers to an aromatic ring system wherein two hydrogen atoms were removed thus having two open valencies for bonding. For example, a phenylene or a phenylene ring system fused to one or more aromatic rings to form, for example, derivatives of anthracene, phenanthrene, or napthalene ring systems.

The term “heteroarylene” refers to an aromatic ring system wherein at least one of the carbon atoms of the aromatic ring system is replaced by a heteroatom (N, O, P, S) and wherein two hydrogen atoms were removed thus having two open valencies for bonding.

The present invention relates to pharmaceutical compositions comprising a compound of the subject invention in admixture with pharmaceutically acceptable auxiliaries, and optionally other therapeutic agents. The auxiliaries must be “acceptable” in the sense of being compatible with the other ingredients of the composition and not deleterious to the recipients thereof.

Pharmaceutical compositions include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association compounds used in the invention or combinations thereof with any auxiliary agent. The auxiliary agent(s), also named accessory ingredient(s), include those conventional in the art, such as carriers, fillers, binders, diluents, disintegrants, lubricants, colorants, flavoring agents, anti-oxidants, and wetting agents.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

The invention further includes a pharmaceutical composition, as hereinbefore described, in combination with packaging material, including instructions for the use of the composition for a use as hereinbefore described.

For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulizers or insufflators.

The exact dose and regimen of administration of the composition will necessarily be dependent upon the therapeutic or nutritional effect to be achieved and may vary with the particular formula, the route of administration, and the age and condition of the individual subject to whom the composition is to be administered.

As used herein, the term “effective amount” means that amount of a drug or pharmaceutical composition that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, slowing the progression of, or a decrease in the rate of advancement of a disease or disorder, condition or symptom. The term also includes within its scope amounts effective to enhance normal physiological function.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 and FIG. 2 show the results of an assay illustrating the protection against oxidative injury by compounds as defined herein above—in mammalian NIH cells.

FIG. 3 and FIG. 4 show the results of survival assay, illustrating the protection against Paraquat (PQ) by compounds as defined herein above—in C. elegans.

FIG. 5 shows the results of proliferation assay of human liver cancer cells (HCC line HUH7), illustration the complete inhibition of multiplication of such cells by a compound as defined herein above.

FIG. 6 shows the scheme for synthesizing ST-910.

FIG. 7 shows the scheme for synthesizing ST-920.

FIG. 8 shows the scheme for synthesizing 932-iso.

FIG. 9 shows the scheme for synthesizing 932.

FIG. 10 shows the scheme for synthesizing 922, 923 and 924.

FIG. 11 shows the scheme for synthesizing 911-Me.

FIG. 12 shows the scheme for synthesizing 912-Me and 914-Me.

FIG. 13 shows the scheme for synthesizing 913-Me.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Synthesis of Compounds of the Invention

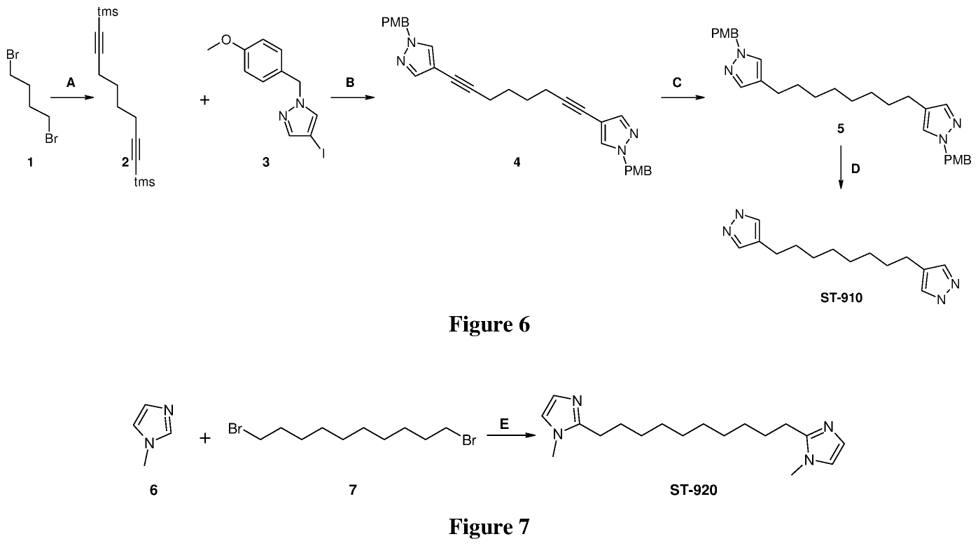

### Synthesis of ST-910 (FIG. 6) and ST-920 (FIG. 7)

Step A: TMS-acetylene (46 mL, 370.4 mmol, 4 eq.) was dissolved in dry THF (400 mL) under argon atmosphere. The solution was cooled to −78° C. and n-BuLi (2.5 M in hexane, 130 mL, 370.4 mmol, 4 eq.) was added dropwise and the resulting mixture was stirred at −78° C. for 15 minutes. The reaction mixture was warmed slowly to 0° C. and stirred for 1 hour. The solution of compound 1 (20 g, 92.6 mmol, 1 eq.) in HMPA (227 mL, 1481.6 mmol, 16 eq.) was added dropwise and the resulting mixture was left to stir at r.t. for 16 h. The resulting mixture was poured into 1 N HCl and extracted with EtOAc (2×400 mL). The combined organic extract was washed with water (2×200 mL), brine (200 mL), dried over sodium sulfate and evaporated to obtain crude 2 (21 g) which was used in next step without purification.

Step B: Compound 3 (29 g, 92.2 mmol, 2.1 eq.) was placed in 1 L 3-necked flask, equipped with argon inlet, thermometer and dropping funnel. The flask was evacuated and backfilled with argon (three cycles). Dry THF (400 mL) was added, followed by compound 2 (11 g, 43.9 mmol, 1 eq.), triethylamine (38.5 mL, 263.4 mmol, 6 eq.), CuI (2.51 g, 13.2 mmol, 0.3 eq.) and Pd(PPh₃)₄ (5.07 g, 4.4 mmol, 0.1 eq.). The resulting mixture was heated to 55° C. TBAF (1 M in THF, 92 mL, 87.8 mmol, 2 eq.) was added dropwise and the resulting mixture was stirred for 16 h at the same temperature. The reaction mixture was cooled to r.t. and evaporated under reduced pressure. The residue was partitioned between EtOAc (400 mL) and water (400 mL). The organic layer was collected, washed with water (4×400 mL), brine (200 mL), dried over sodium sulfate and evaporated under reduced pressure. The residue was subjected to column chromatography on SiO₂ to obtain compound 4 (10 g).

Step C: Compound 4 (10 g, 20.9 mmol) was hydrogenated at atmospheric pressure, using 10% Pd/C (1 g) as catalyst for 24 h. All insoluble materials were filtered off. The filtrate was evaporated to dryness to obtain compound 5 (10 g), which was used in next step without purification.

Step D: Compound 5 (10 g, 20.8 mmol) was mixed with anisole (50 mL). TFA (100 mL) was added and the resulting mixture was heated at 115° C. (oil bath temperature) under argon atmosphere for 10 days (determination of the presence of starting material by NMR reaction mixture). The resulting dark mixture was cooled to r.t. and evaporated under reduced pressure. The residue was subjected to column chromatography to obtain ST-910 (0.5 g, bis-trifluoroacetate salt).

Step E: To a stirred suspension of N-methylimidazole (6) (10 g, 120 mmol, 2 eq.) in diethyl ether (600 mL) at −70° C. was added n-butyl lithium (2.5 M in THF, 49 mL, 120 mmol, 2 eq.). The mixture was allowed to warm to −10° C. and maintained at this temperature for 30 min. The white suspension was cooled to −70° C. and 1,10-dibromodecane (7) (18 g, 60 mmol, 1 eq.) was added. The solution was allowed to warm to ambient temperature and stirred for 16 h. The reaction mixture was poured into water (200 mL) and extracted with ether (2×400 mL). The combined organic extract was evaporated to dryness. The residue was partitioned between EtOAc (200 mL) and 1 N HCl (100 mL). The organic layer was discarded. The aqueous layer was evaporated to dryness and the residue was dissolved in 50% aq. NaOH (20 mL). The resulting mixture was extracted with ether (2×50 mL). The combined organic extract was evaporated under reduced pressure. The residue was subjected to column chromatography to obtain ST-920 (2.3 g).

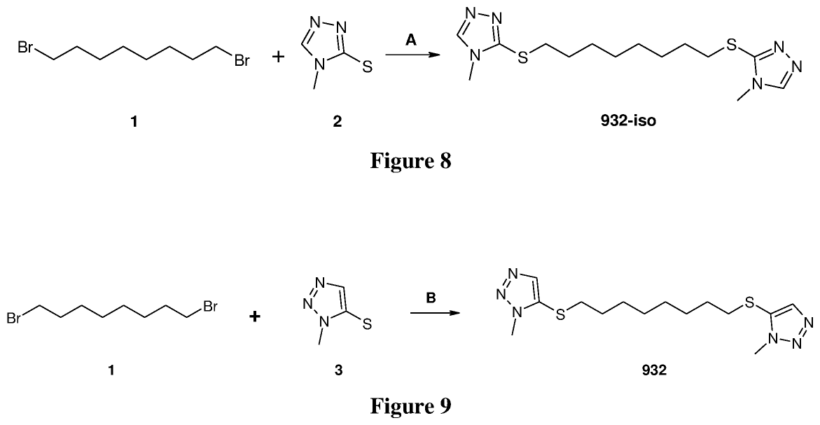

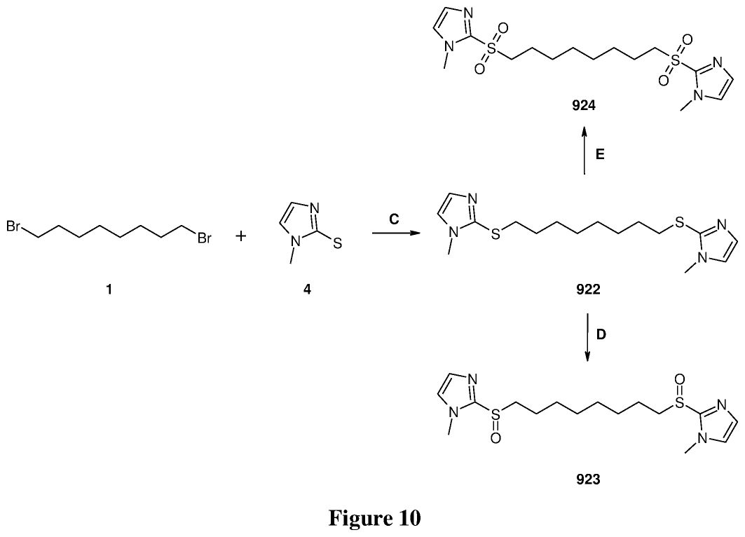

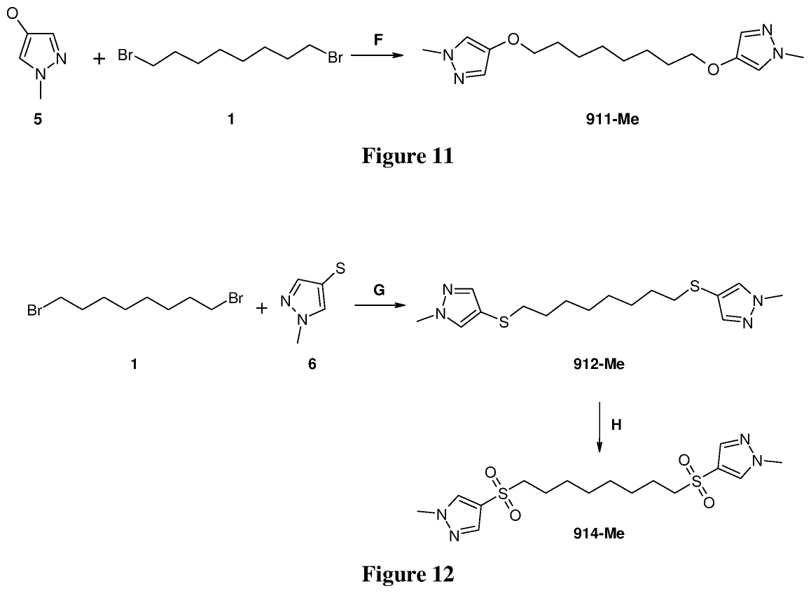

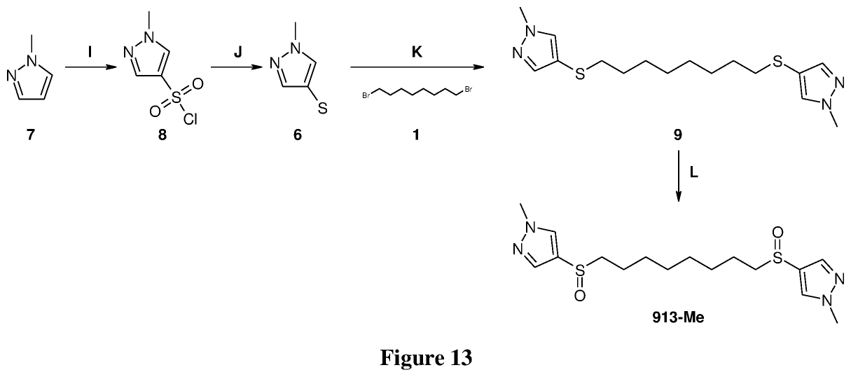

### Synthesis of 932-iso (FIG. 8), 932 (FIG. 9), 922, 923, 924 (FIG. 10), 911-Me (FIG. 11), 912-Me, 914-Me (FIG. 12), 913-Me (FIG. 13)

Step A: To a solution of compound 2 (4.25 g, 2 eq.) in MeOH (45 mL) at 0° C. was added NaOH (15% solution in water, 2 eq.). After 30 min compound 1 (5 g, 1 eq.) was added to the reaction mixture at the same temperature. The resulting solution was stirred at r.t. overnight. The next day the mixture was concentrated under reduced pressure, diluted with water and the precipitate formed was collected by filtration and dried to afford compound 932-iso (3 g).

Step B: To a solution of compound 3 (4.23 g, 2 eq.) in MeOH (45 mL) at 0° C. was added NaOH (15% solution in water, 2 eq.). After 30 min compound 1 (5 g, 1 eq.) was added to the reaction mixture at the same temperature. The resulting solution was stirred at r.t. overnight. The next day the mixture was concentrated under reduced pressure, diluted with water and extracted with EtOAc (2×40 mL). The organic layers were combined and concentrated under reduced pressure to afford the crude product, which was purified with flash column chromatography to afford compound 932 (2 g).

Step C: To a solution of compound 4 (4.2 g, 2 eq.) in MeOH (45 mL) at 0° C. was added NaOH (15% solution in water, 2 eq.). After 30 min compound 1 (5 g, 1 eq.) was added to the reaction mixture at the same temperature. The resulting solution was stirred at r.t. overnight. The next day the mixture was concentrated under reduced pressure, diluted with water and the precipitate formed was collected by filtration and dried to afford compound 922 (3.2 g).

Step D: To a solution of compound 922 (27 g, 80 mmol.) in ethyl acetate (900 mL), cooled to −5° C., was added m-CPBA (34 g, 80% purity). The resulting solution was stirred at r.t. overnight. After that a sat. aq. solution of Na₂S₂O₃ (100 mL) was added to the reaction mixture. The organic layer was separated and concentrated under reduced pressure to afford the crude product, which was purified with flash column chromatography and then washed with Et₂O to obtain 10 g of compound 923.

Step E: To a solution of compound 922 (4 g, 1 eq.) in dichloromethane (80 mL) was added m-CPBA (12 g, 6 eq.) at 0° C. The resulting solution was stirred at r.t. overnight. The next day the mixture was washed with a sat. aq. solution of NaHCO₃ (4×80 mL). The organic layer was separated and concentrated under reduced pressure to afford the crude product, which was purified with flash column chromatography to obtain 2 g of compound 924.

Step F: To a solution of compound 5 (7.75 g, 79 mmol) in DMA (50 mL) was added in portions NaH (3.5 g, 82.6 mmol) and the mixture was stirred for 1 h at r.t. After that, compound 1 (9.8 g, 35.9 mmol) was added and the reaction mixture was stirred for 16 h at 70° C., cooled to r.t. and poured into water. The precipitated solid was collected, washed with water, isopropanol and hexane, and dried at 50° C. to afford 2.56 g of compound 911-Me (23%).

Step G: To a solution of compound 6 (4 g, 1 eq.) in CH₃CN (60 mL) at 10° C. was added K₂CO₃ (2 eq.). After 30 min of stirring at room temperature compound 1 (4.7 g, 1 eq.) was added to the reaction mixture at r.t. The resulting solution was stirred at r.t. overnight. The next day the mixture was concentrated under reduced pressure, diluted with water and the precipitate formed was collected by filtration and dried to afford compound 912-Me (3.2 g).

Step H: To a solution of compound 912-Me (3.2 g, 1 eq.) in dichloromethane (80 mL) was added m-CPBA (10 g, 5 eq.) at 10° C. The resulting solution was stirred at r.t. overnight. The next day the mixture was washed with a sat. aq. solution of NaHCO₃ (4×80 mL). The organic layer was separated and concentrated under reduced pressure to afford the crude product, which was purified with flash column chromatography to obtain 2.1 g of compound 914-Me.

Step I: To stirred chlorosulfonic acid (91 mL, 1220 mmol) at 5° C. was added compound 7 (25 g, 304 mmol) over 1 hour. After the addition was completed the reaction mixture was stirred at ambient temperature for 30 min and then heated at 110 C for 16 h. The reaction mixture was cooled to 5° C. and thionyl chloride (55 mL, 754 mmol) was added dropwise and the resulting mixture was stirred at ambient temperature for 1 h and heated under reflux for 2 h. The reaction mixture was cooled to 15 C and carefully quenched into crushed ice. The solid was collected by filtration and washed with water to obtain 46 g of compound 8.

Step J: Compound 8 (46 g, 255 mmol) was dissolved in toluene (1500 mL), and to the solution was added triphenylphosphine (200 g, 764 mmol) in portions. The reaction mixture was stirred at room temperature overnight, then filtered and evaporated under reduced pressure. To the residue was added hexane, the solid was separated and the solvent was evaporated under reduced pressure. Pure compound 6 (14 g) was obtained by distillation under reduced pressure.

Step K: Compound 6 (4.56 g, 40 mmol) was dissolved in EtOH (60 mL), KOH (2.24 g, 40 mmol) was added and the mixture was stirred for 15 min at ambient temperature. After that, compound 1 (5.54 g, 20 mmol) was added. The reaction mixture was refluxed overnight, cooled and concentrated in vacuo. The residue was dissolved in EtOAc, washed with brine, dried over sodium sulfate and the solvent was evaporated under reduced pressure to obtain 6.1 g of compound 9.

Step L: Compound 9 (3 g, 8.9 mmol) was dissolved in EtOAc (120 mL), cooled to −5° C. and m-CPBA (3.82 g, 80% purity) was added in small portions. The reaction mixture was stirred overnight, then a saturated solution of Na₂S₂O₃ (20 mL) was added. The organic layer was separated and concentrated in vacuo. The residue was purified by column chromatography to obtain 1.3 g of 913-Me. The reaction was repeated twice.

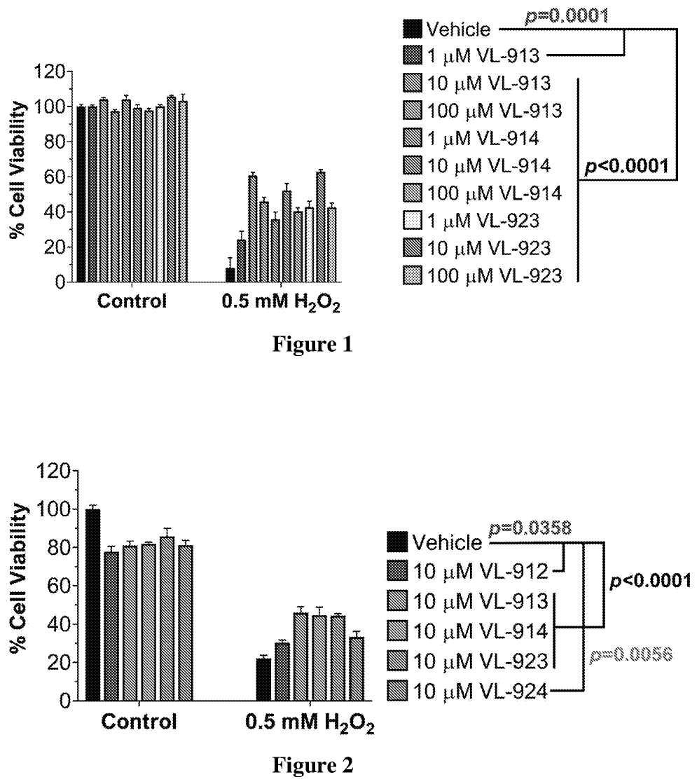

Oxidative injury assays—in mammalian NIH cells: NIH cells were seeded in 96-well plates at a density of 7,500 and 12,000 cells per well, in 100 μl DMEM high glucose media without phenol red supplemented with 2% L-glutamine, 1% tetracycline, 10% heat inactivated FBS. The cells were incubated at 37° C., 5% CO₂. After 24 hrs, when cells showed 50-60% confluence, the drugs were added and vehicle and incubate d the cells under the same conditions for additional 24 hrs. To induce oxidative injury, H₂O₂ was added to a final concentration of 0.5 mM, and incubated the cells for 3 hrs. Then, the cells were washed three times with 100 μl PBS and were supplemented with 100 μl of fresh medium that did not contain any drug. We incubated the cells for 24 hrs, as before, and measured their viability using XTT assay according to the manufacturer instructions. FIGS. 1 and 2 provide the results.

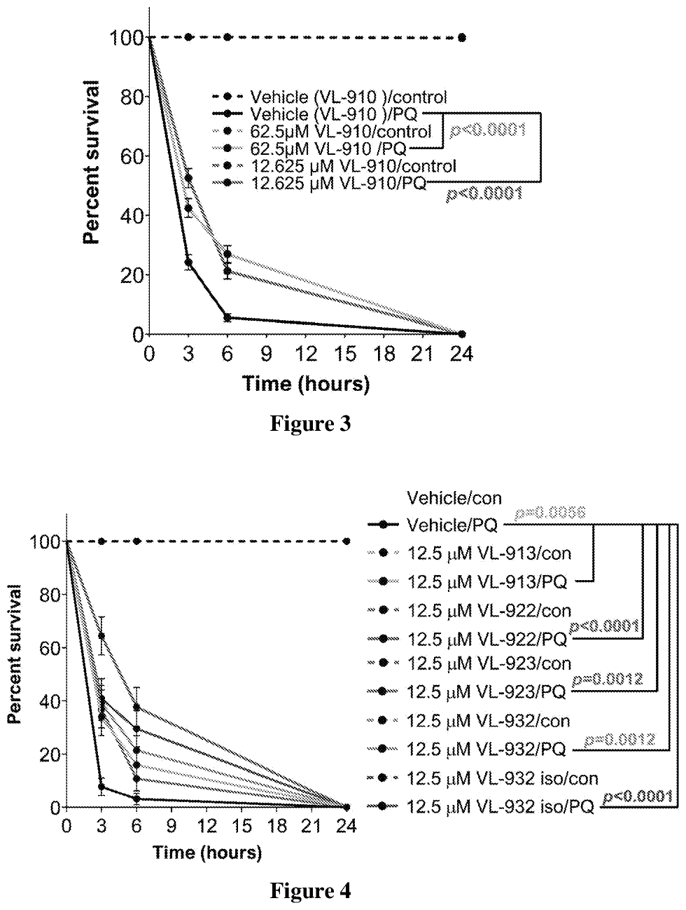

Paraquat (PQ) resistance assay in C. elegans: Paraquat assays were conducted as described in (Romero-Afrima, L., Zelmanovich, V., Abergel, Z., Zuckerman, B., Shaked, M., Abergel, R., Livshits, L., Smith, Y., and Gross, E. (2020). Ferritin is regulated by a neuro-intestinal axis in the nematode Caenorhabditis elegans. Redox Biology 28) with some modifications. Synchronized ˜200 L1 larvae were grown in 35 mm seeded NGM plates supplemented with indicated drug concentrations or with vehicle as a control until the desired developmental stage. The worms were collected from the plate with M9 buffer, washed twice with the same buffer, and transferred into a 96-well plate (˜12 worms per well, 100 μl). PQ (200 mM, final concentration) or M9 (control) were added to each well, and the plate was placed on an orbital shaker at 350 rpm at 21° C. An eyelash pick was used to score worms' survival (each worm was touched several times over the course of a few seconds) when PQ was added (time 0) and 3, 6 and 24 hrs afterward. Six biological repeats with a minimum of 120 worms per condition were performed. FIGS. 3 and 4 provide the results.

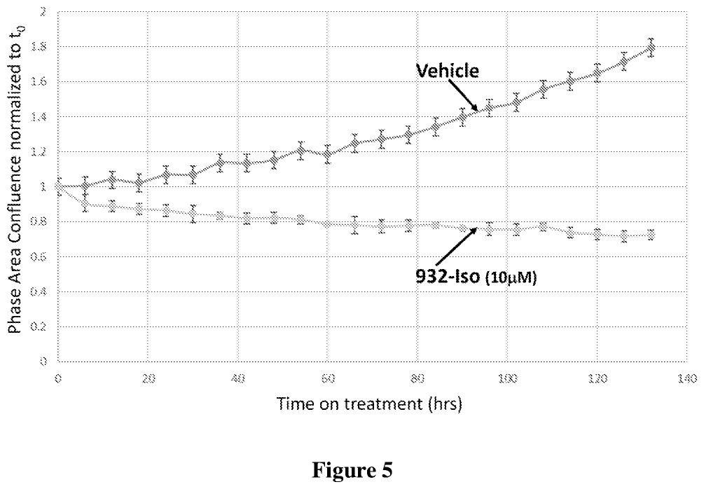

Proliferation assay of human HCC line HUH7: Cells were seeded in a 48-well tissue-culture plate, 2,000 cells per well, in a final volume of 0.5 ml medium (DMEM supplemented with 100 IU/ml penicillin, 100 mg/ml streptomycin, and 2% L-glutamine+10% FCS). After letting the cells settled for 24 h, either DMSO at a final concentration of 0.1% (=Vehicle) or above described compound 932-Iso at a final concentration of 10 μM, including 0.1% DMSO, were added to the cells, 4 well replicates for each system. The kinetics of cell proliferation was then monitored utilizing the IncuCyte detection system (Essen BioScience, for details see: Artymovich K, Appledorn D M. A Multiplexed Method for Kinetic Measurements of Apoptosis and Proliferation Using Live-Content Imaging. Methods Mol Biol. (2015) 1219: 35-42). The results are depicted in FIG. 5.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A compound having a general formula (I)
R₁—X₁-L-X₂—R₂ (I)
wherein R₁ and R₂ are each independently selected from —C(═NR₃)NR₄R₅, —NR₆R₇, —N⁺R₈R₉R₁₀, —NR₁₁C(═N)NR₁₂R₁₃, —NR₁₈NR₁₉R₂₀, —ONR₂₂R₂₃, —NR₁₄C(═N)—NR₁₅—C(═N)—NR₁₆R₁₇═N—R₂₁ 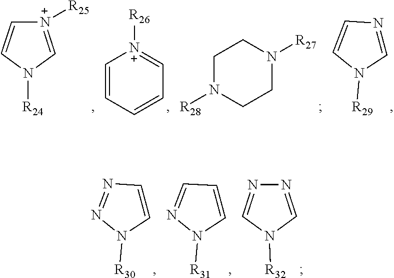
wherein each of R₃-R₃₂ is independently selected from H, straight or branched C₁-C₁₂ alkyl, straight or branched C₂-C₁₂ alkenyl, straight or branched C₂-C₁₂ alkynyl, phenyl, —OH, halogen and any combinations thereof,
L is selected from straight or branched C₄-C₁₂ alkylene, straight or branched C₄-C₁₂ alkenylene, straight or branched C₄-C₁₂ alkynylene;
each L is optionally interrupted by at least one of C₄-C₈ cycloalkylene, C₄-C₈ cycloalkenylene, C₄-C₈ cycloalkynylene, arylene, heteroarylene, heteroatom and any combinations thereof;
each L is optionally substituted with at least one of halogen and any combinations thereof;
X₁ and X₂ are independently selected from null, —O—, —S—, —S(═O)—, —S(═O)₂—; provided that when X₁ and X₂ are each selected from null, —O—, —S— than R₁ and R₂ are independently selected from

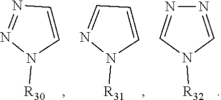

2. A compound according to claim 1, wherein L is straight or branched C₄-C₁₂ alkylene.

3. A compound according to claim 1, wherein L is interrupted by at least one of C₄-C₈ cycloalkylene, C₄-C₈ cycloalkenylene, C₄-C₈ cycloalkynylene, aryl, heteroaryl, heteroatom and any combinations thereof.

4. A compound according to claim 1, wherein L is interrupted by at least one C₄-C₈ cycloalkylene.

5. A compound according to claim 1, wherein L is interrupted by at least one C₄-C₈ cycloalkenylene.

6. A compound according to claim 1, wherein L is interrupted by at least one C₄-C₈ cycloalkynylene.

7. A compound according to claim 1, wherein L is interrupted by at least one aryl selected from phenyl or biphenyl.

8. A compound according to claim 1, wherein L is interrupted by at least one heteroaryl.

9. A compound according to claim 1, wherein L is interrupted by at least one heteroatom selected from N, O, S.

10. (canceled)

11. (canceled)

12. (canceled)

13. (canceled)

14. (canceled)

15. A compound according claim 1, wherein R₁ and R₂ are each —C(═NR₃)NR₄R₅.

16. A compound according to claim 1, wherein R₁ and R₂ are each selected from —NR₆R₇ and —N⁺R₈R₉R₁₀.

17. A compound according to claim 1, wherein R₁ and R₂ are each selected from —NR₁₁C(═N)NR₁₂R₁₃ and —NR₁₄C(═N)—NR₁₅—C(═N)—NR₁₆R₁₇.

18. A compound according to claim 1, wherein R₁ and R₂ are each —NR₁₈NR₁₉R₂₀.

19. A compound according to claim 1, wherein R₁ and R₂ are each ═N—R₂₁.

20. A compound according to claim 1, wherein R₁ and R₂ are each —ONR₂₂R₂₃.

21. A compound according to claim 1, wherein R₁ and R₂ are each selected from

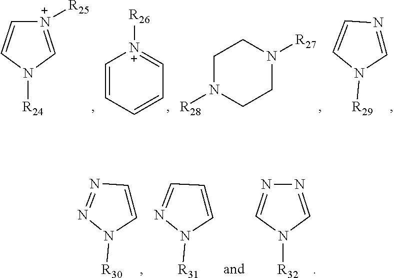

22. (canceled)

23. (canceled)

24. (canceled)

25. (canceled)

26. (canceled)

27. (canceled)

28. A compound according to claim 1, wherein X₁ and X₂ are each independently selected from null, —O—, —S— and any combinations thereof.

29. A compound according to claim 1, wherein X₁ and X₂ are each independently selected —S(═O)—, —S(═O)₂— and any combinations thereof.

30. (canceled)

31. (canceled)

32. (canceled)

33. (canceled)

34. (canceled)

35. (canceled)

36. (canceled)

37. A method of at least one of (i) maintaining the vitality of non-regenerating tissue; (ii) tretment of a condition or a disease associated with cell degeneration; (iii) treatment or prevention of a disease, disorder, symptom, which is caused by, associated with, or aggravated by impaired mitophagy, oxidative injury or oxidative stress; in a subject, said method comprising administering to said subject an effective dose of a compound having a general formula (I)
R₁—X₁-L-X₂—R₂ (I)
wherein R₁ and R₂ are each independently selected from —C(═NR₃)NR₄R₅, —NR₆R₇, —N⁺R₈R₉R₁₀, —NR₁₁C(═N)NR₁₂R₁₃, —NR₁₈NR₁₉R₂₀, —ONR₂₂R₂₃, —NR₁₄C(═N)—NR₁₅—C(═N)—NR₁₆R₁₇═N—R₂₁ 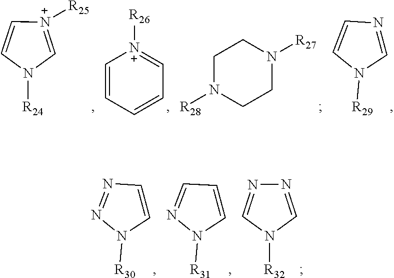
wherein each of R₃-R₃₂ is independently selected from H, straight or branched C₁-C₁₂ alkyl, straight or branched C₂-C₁₂ alkenyl, straight or branched C₂-C₁₂ alkynyl, phenyl, —OH, halogen and any combinations thereof;
L is selected from straight or branched C₄-C₁₂ alkylene, straight or branched C₈-C₁₂ alkenylene, straight or branched C₄-C₁₂ alkynylene;
each L is optionally interrupted by at least one of C₄-C₈ cycloalkylene, C₄-C₈ cycloalkenylene, C₄-C₈ cycloalkynylene, arylene, heteroarylene, heteroatom and any combinations thereof;
each L is optionally substituted with at least one of halogen and any combinations thereof;
X₁ and X₂ are independently selected from null, —O—, —S—, —S(═O)—, —S(═O)₂—.

38. (canceled)

39. (canceled)

40. (canceled)

41. (canceled)

42. (canceled)

43. A method according to claim 37, wherein said condition or a disease associated with cell degeneration is selected from Parkinson's disease, Alzheimer's disease, dementia, congestive heart failure, sarcopenia, type 2 diabetes, age-related macular degeneration (AMD), atherosclerosis, cardiovascular diseases, cancer, liver diseases, pancreatic diseases, ocular diseases, arthritis, cataracts, osteoporosis, hypertension, fibrosis, including lung-fibrosis, and any combinations thereof.

44. (canceled)

45. (canceled)

46. (canceled)

47. (canceled)
